(19) United States
(12) Patent Application Publication
(10) Pub. No.: US 2007/0184554 A1
(43) Pub. Date: Aug. 9, 2007

(54) Biomarker of improved intestinal function

(75) Inventors: Nathan S. Teuscher, Lincoln, NE (US); Lidia L. Demchyshyn, Toronto (CA); David S. Wells, Salt Lake City, UT (US)

Correspondence Address: Connolly Bove Lodge & Hutz LLP, Suite 800, 1990 M Street, N.W., Washington, DC, 20036, US

(73) Assignee: NPS ALLELIX CORP., Toronto, CA (US)

(21) Appl. No.: 11/606,896

(22) Filed: Dec. 1, 2006

Related U.S. Application Data
(60) Provisional application No. 60/741,074, filed on Dec. 1, 2005.

(51) Int. Cl. G01N 33/00

(52) U.S. Cl. 436/89

(57) ABSTRACT

The invention disclosed herein demonstrates that that the adaptive process in the intestine can be tracked using plasma citrulline. It further demonstrates that plasma citrulline is of clinical utility as a biomarker for improvements in intestinal function.

### CROSS-REFERENCE TO RELATED APPLICATION

This application claims priority under 35 U.S.C. § 119(e) to U.S. Provisional Application No. 60/741,074 filed on Dec. 1, 2005, the entire contents of which are hereby incorporated by reference.

### FIELD OF THE INVENTION

The present invention relates to a biomarker of intestinal function and to the use of the biomarker in the monitoring of improvements in intestinal function and in the monitoring of the adaptive response of the intestine as a result of drug treatment or other therapy.

### BACKGROUND OF THE INVENTION

Glucagon-like peptide 2 (GLP-2), a 33-amino acid peptide that arises from tissue specific processing of the glucagon precursor, proglucagon, within the mucosal L-cells of both the small and large bowel and the specific neurons located in the brain stem (Drucker, 2002, Gut.50:428-435; Burrin et al., 2003, Domest. Anim Endocrinol. 24:103-122,). GLP-2 is co-secreted with GLP-1 from the intestinal enteroendocrine L-cells, with the presence of luminal nutrients being the primary stimulus for secretion. Circulating levels of GLP-2 rises rapidly after ingestion of nutrients and the intact peptide is rapidly degraded to an inactive metabolite, GLP-2 (3-33), via the enzyme dipeptidyl peptidase IV (DPP IV)(Hartmann et al. 1997, J Clin. Endocrin. 85:2884-2888). Recent data have indicated a strong positive correlation between circulating GLP-2 concentration and intestinal mucosal growth (Burrin et al., 2000, Am J Clin Nutr. 71:1603-1610). In addition to its potent trophic effects on the intestinal mucosa, GLP-2 inhibits gastric emptying (Wettergren et al. 2004, Scand. J. Gastroenterol. 39(4): 353-358) and gastric acid secretion, stimulates intestinal barrier function, stimulates intestinal hexose transport, and enhances nutrient absorption in rodents and in human patients with short bowel syndromes (SBS) (Drucker et al., 2004, Endocrinology, 146:19-21). GLP-2 exerts it action through binding to the GLP-2 receptor, a G-protein coupled receptor most closely related to the GLP-1 and glucagon receptors of the secretin family of receptors (Monroe et al. 1999, Proc. NatL. Acad. Sci. USA 96: 1569-1573). The GLP-2 receptor is linked to the activation of adenylyl cyclase and hence cAMP formation. The receptor appears to be predominantly expressed in the gut, particularly the intestines and in the compact part of the dorsomedial hypothalamic (DMH) nucleus (Larsen et al., 2000, Nature Medicine 6(7):802-807, Orskov et al., 2005, Regulatory Peptides. 124:105-112).

The process of intestinal adaptation following organ insult has been widely studied, with reports as early as the 19^{th} century (Senn, 1888; Flint, 1912). More recently a variety of measures of intestinal status have been used to assess intestinal adaptation in a quantitative manner. These measures include villus height and crypt depth (Scott et al., 1998; Drucker, 2002; Hartmann et al., 2002; Perez et al., 2005), proliferative index (Perez et al., 2005), apoptotic index (Hartmann et al., 2002; Perez et al., 2005), luminal surface area (Ljungmann et al., 2001), volume fractions (Ljungmann et al., 2001), tissue weight (Scott et al., 1998; Ljungmann et al., 2001; Hartmann et al., 2002; Lardy et al., 2004), tissue length (Scott et al., 1998; Hartmann et al., 2002; Lardy et al., 2004), transporter/enzyme activity assays (Scott et al., 1998; Drucker, 2002; Lardy et al., 2004), and DNA content (Scott et al., 1998). All of these measures have a quantitative component, yet each suffers from potential bias due to the lack of specificity in describing the overall adaptive process in the intestine. The villus height and crypt depth measurements are made on individual sections of tissue that may not represent the entire organ or even the entire tissue sample. Histological measures of proliferation and apoptosis suffer the same limitation as only a small portion of the tissue can be evaluated. Gross pathology, such as tissue weight and length, are dependent upon an unknown starting point for a given animal, do not specifically address function, and are less sensitive than desired for small changes. Transporter assays and DNA content evaluate only portions of the intestine, and do not capture the complete role of the organ. In most cases several of these measures are implemented in a study to describe the adaptive process because a single measure is insufficient.

Plasma citrulline is an endogenous amino acid that is not incorporated into peptides. Early amino acid metabolism work suggested that circulating citrulline was a precursor of arginine (Windmueller and Spaeth, 1981), while work on the urea cycle identified citrulline as an intermediate in the nitrogen metabolism pathway (Felig and Wahren, 1971; Windmueller and Spaeth, 1974; Windmueller and Spaeth, 1980). This work also established that enterocytes lack the mitochondrial enzymes of the urea cycle that convert citrulline into arginine (Windmueller and Spaeth, 1981). Windmueller and Spaeth concluded that the intestine is the primary source of circulating citrulline, while the kidney is responsible for uptake and conversion into arginine (Windmueller and Spaeth, 1981). And importantly, the liver played no role in citrulline uptake or release, suggesting that the intestinal-renal pathway accounts for the majority of citrulline turnover, and controls steady-state levels in the body.

Recently, several clinicians studying small bowel diseases, such as short bowel syndrome (Crenn et al., 1998; Wasa et al., 1999b; Wasa et al., 1999a; Jianfeng et al., 2005; Rhoads et al., 2005), villous atrophy (Crenn et al., 1998; Crenn et al., 2003), and chemotherapy-induced mucosal atrophy (Lutgens et al., 2004; Lutgens et al., 2005) began measuring amino acid levels to evaluate nutritional status of their patients. In all cases, small intestine damage was associated with below normal plasma citrulline levels. In particular, plasma citrulline levels are in agreement with the kinetics of epithelial loss following radiotherapy (Lutgens et al., 2005) and are useful in categorizing patients with permanent intestinal failure (Crenn et al., 1998).

Biomarkers

The idea that specific physiologic measures can predict clinical outcomes is not a new concept. For example, changes in blood glucose levels are used to monitor diabetes; blood pressure measurements are used to assess heart function; specific algorithms are used to assess the blood clotting potential (International Normalized Ratio or INR) of blood thinning agents such as warfarin. Thus, when diminished capacity is associated with a decreased physiologic measure, most scientists logically assume that improvement in the physiologic measure must mean improved capacity. Although logical, this is not always the case. In fact, more often the relationship between the disease and the physiologic marker is more complex, and improvements in the physiologic marker are not associated with improved capacity. Two specific examples of this phenomenon are the association between heart arrhythmias and mortality and the association between bone mineral density and fracture risk.

The Cardiac Arrhythmia Suppression Trial (CAST) was designed to evaluate the ability of encainide and flecainide to reduce arrhythmia incidence. Prior to the study, a direct correlation between increased arrhythmias and increased mortality was reported. Therefore, the investigators believed that a reduction in the number of arrhythmias would result in improved survival. Although both drugs produced a significant reduction in arrhythmia incidence, mortality was 3-fold greater in the drug treatment groups. Thus a decrease in arrhythmias was not correlated with improved survival, even though an increase is correlated with mortality.

Osteoporosis is a disease characterized by significant bone loss and weakening of the bones, resulting in fractures. A decrease in bone mineral density was associated with increased fracture risk and increased bone loss. The administration of fluoride resulted in significant increases in bone mineral density. However, this was associated with increased fracture risk and weaker bone strength. Therefore, although the decrease in BMD was correlated with increased fracture risk, an increase in BMD did not result in decreased fracture risk.

Both of these examples suggest that bidirectional correlations between biomarkers and clinical outcomes are not obvious. In part, this is because some biomarkers may have correlations because of chance rather than specific physiologic rationale. In addition, the biomarkers may only correlate in one direction because they are not reversible events. In conclusion, although bidirectional correlations are logical, the demonstration of that relationship is novel and non-obvious.

### SUMMARY OF THE INVENTION

We have found that plasma citrulline is a biomarker for improvements in intestinal function. Thus, one aspect of the invention disclosed herein is an assay for determining improvements in intestinal function in an individual by measuring the level of plasma citrulline. Another aspect of the invention is a method for monitoring the level of intestinal function in an individual. Yet another aspect of the invention is a method for monitoring the adaptive process of the intestine in an individual by monitoring the level of plasma citrulline over time. A further aspect of the invention is monitoring the adaptive process of the intestine in an individual undergoing treatment with an analogue of GLP-2.

A further aspect of the invention is monitoring the protection or restoration of mucosal integrity as a result of a challenge such as disease or medical treatment resulting in intestinal damage.

Another aspect of the invention is in the assessment of novel therapies (or novel treatment regimens) for the treatment of intestinal damage in both human and non-human animals (e.g. in animal models).

Another aspect of the invention is in the assessment of the appropriate dosage level for the treatment of intestinal damage in both human and non-human animals (e.g. in animal models).

A further aspect of the invention is a diagnostic or monitoring kit for carrying out the method of the invention.

### BRIEF DESCRIPTION OF THE FIGURES

FIG. 1 shows the plasma citrulline concentration-time data for all 5 treatment groups in the animal studies.

FIG. 2 shows box and whisker plots for plasma citrulline levels measured as part of a clinical study into the treatment of Crohn's Disease using Teduglutide in humans.

### DETAILED DESCRIPTION OF THE INVENTION

The intestinal adaptation process has been studied intensively for over 100 years, yet, even today, the tools used to characterize the adaptive process are complicated and potentially biased. One well-established model system used to study intestinal adaptation is that of small bowel resection in rats. The time course of the adaptive process in rats is well characterized in this model, with complete adaptation occurring within 30 days following intestinal resection (Dowling and Booth, 1967; Hanson et al., 1977). These estimates have been obtained using invasive techniques that require multiple animals to be sacrificed at each time point, denying the opportunity to observe the progression of an individual animal. Furthermore, these invasive techniques are inappropriate for a clinical study.

For the first time, a pharmacodynamic model of plasma citrulline as a marker for intestinal function and adaptation has been developed. The exponential growth model was fit to plasma citrulline concentration-time data from rats that underwent small bowel resection surgery. The results show that plasma citrulline is a biomarker for intestinal function and correlates with the adaptive process.

The invention disclosed herein clearly demonstrates that plasma citrulline responds to intestinal resection in the same time frame as the adaptive process in this model system. The surgery causes an initial decrease in circulating plasma citrulline followed by a growth phase until a new steady-state is achieved approximately 15-25 days after the surgery. In effect, plasma citrulline levels track the adaptive process and, thus, can be used to monitor the adaptive response in a much less invasive manner than techniques of the art.

The invention disclosed herein has utility, for example, in the monitoring of patients undergoing therapy to improve intestinal function which has been compromised, for example by disease (such as Crohn's Disease), or as a result of chemo—or radiation-therapy to treat cancer. These patients can be monitored in a much less invasive, and more effective, manner than has been possible in the past. In practice, the method comprises the steps of (a) determining the level of plasma citrulline in a subject at a first time point (for example, before the start of treatment); (b) treating the subject and (c) determining the level of plasma citrulline in a subject at a second and, optionally, subsequent time points

In another example, patients undergoing treatment with an analogue of GLP-2 (such as Teduglutide) can be monitored to track their response to treatment and to modify, if necessary, the treatment regimen to ensure optimum results. An analogue of GLP-2 is defined as any molecule which interacts with the GLP-2 receptor; such molecules may be peptides (such as analogues of naturally occurring GLP-2) or small molecules.

A further aspect of the invention is monitoring the protection or restoration of mucosal integrity as a result of a challenge such as disease or medical treatment resulting in intestinal damage.

With respect to the animal data disclosed herein, the sham group provided significant information on the sensitivity of plasma citrulline to minor intestinal injury. The partial transection resulted in an immediate decrease in plasma citrulline levels on Day 2. However within 1 week the plasma citrulline levels had returned to the pre-surgery levels. The fact that plasma citrulline levels rapidly returned to pre-surgery levels in the sham animals suggests that no intestinal function was lost following the partial transection. It is thought that a reduction in citrulline production occurs as the animal makes an effort to repair the transected intestinal segment. Following repair, resources are then reallocated to citrulline production. The transient nature of the citrulline response in the sham group suggests that it is a sensitive marker of intestinal function and that citrulline responds to intestinal finction changes within 24 hours.

The plasma citrulline response for the treated animals (i.e. following small bowel resection) was quite different from the sham animals. Following small bowel resection, all animals experienced a significant drop in the plasma citrulline levels. The 50% and 80% resections averaged a loss of 67% and 70%, respectively, of the plasma citrulline within 24 hours of the surgery. This decrease was immediate, yet transient, as plasma citrulline levels started to increase on Day 3 indicating that adaptation begins almost immediately following surgery. Previous work in animals and humans developed correlations of intestinal function and plasma citrulline levels after reaching steady-state (Crenn et al., 1998; Wasa et al., 1999b; Wasa et al., 1999a; Crenn et al., 2000; Crenn et al., 2003; Lutgens et al., 2003; Lutgens et al., 2004; Jianfeng et al., 2005; Lutgens et al., 2005; Rhoads et al., 2005). The lack of a correlation between the minimum citrulline levels and the extent of the resection suggests that although plasma citrulline responds rapidly, it is a measure of steady-state intestinal function rather than instantaneous intestinal function. Furthermore, decreases in the short term may reflect a redistribution of resources rather than actual damage as demonstrated in the sham groups.

The maximum plasma citrulline levels after adaptation indicate that although a new steady-state is reached, the small intestine does not regain all of the capacity that was lost through surgical resection. In fact, following resection although there is an adaptive response, the loss of intestinal mass remains. Results from other analyses suggest that function is restored following adaptation (Ljungmann et al., 2001); however, plasma citrulline does not return to pre-surgery levels. This suggests that the normal intestine contains excess capacity, which the adaptive process cannot restore. The new steady-state levels were similar for the 50% and 80% resections, regardless of resection location (proximal vs. distal). A 50% small bowel resection in humans often results in supplemental nutrition, whereas an 80% resection in rats is fully recoverable. This occurs because intestinal adaptation in rats occurs to a greater extent and more quickly than observed in humans (Dowling, 1982; Drucker, 2002). We hypothesize that in order to observe differences in post-adaptation steady-state plasma citrulline levels in rats, it would be necessary to conduct small bowel resections (>80%) that require nutritional support.

The growth rate estimates were different when categorized by extent of resection. The 80% resection animals had slower growth rates than the 50% resection animals. This suggests that the adaptive process depends on cellular growth in the intestines. That growth is proportional to the remaining intestinal mass. In fact, the growth rates for the 50% resections are about twice as fast as those observed for the 80% resection groups. One would expect that more severe resections would result in even slower growth rates, as less intestinal mass would be available for adaptation.

The pharmacodynamic model developed to predict plasma citrulline concentrations fit the data well and described the majority of the observed variability. The time course of the observed responses is consistent with the known adaptive process in rats and suggests that plasma citrulline may track intestinal function. Importantly, plasma citrulline sampling is simple and allows an individual animal to be tracked throughout the adaptive process. This makes plasma citrulline an attractive biomarker of intestinal function for use in clinical settings. Further, this model provides a basis from which therapeutic agents and regimens can be tested for their effect on the intestines. Increases in citrulline would suggest improvements in intestinal function. Changes in the maximal response would suggest improvement over the normal adaptive process while changes in the rate may suggest the acceleration of the adaptive process. Pre-surgery regimens could also be evaluated to determine protective effects by attenuating the initial reduction in plasma citrulline.

Pharmacodynamic Analysis

A complete plasma citrulline profile was obtained from the “population” of rats in each treatment group. A mixed-effects modeling approach was utilized to evaluate the plasma citrulline concentration-time data. The mixed-effects modeling approach permits the evaluation of the population parameter estimates, interindividual variability, and residual variability. Furthermore, all four small bowel resection groups can be analyzed simultaneously using a single model. The nonlinear mixed-effects modeling software NONMEM (double precision, version VI.1, UCSF, San Francisco, Calif.) was used for model fitting. The graphical user interface PDx-Pop (Globomax, a division of ICON, Ellicott City, Md.) was employed to execute NONMEM runs and evaluate results. Model selection criteria included a reduction in the objective function value, visual inspection of the population and individual predicted values compared to the observed data, random distribution of the residuals, and examination of residual variability estimates. GraphPad Prism (v4.02, GraphPad Software, San Diego, Calif) was used to generate graphics and statistics.

Prior to this study, no known models existed to predict plasma citrulline concentrations in the rat during the intestinal adaptation process. It was hypothesized that prior to surgery, citrulline is present at a steady-state concentration. Following resection, plasma citrulline levels would decline to a minimum following surgery. Thereafter, it was assumed that an exponential growth model would describe a rise in plasma citrulline levels to either pre-surgery levels or a new steady-state level. Based on these theoretical considerations and the model evaluation criteria mentioned previously, the model used to evaluate the plasma citrulline concentration-time data was divided into two parts. Samples prior to surgery represent steady-state levels of citrulline, and all small bowel resection groups were analyzed together to get a population estimate for Baseline. Following surgery, plasma citrulline concentrations were modeled, by small bowel resection group, as follows:
C(t)=Min+(Max−Min)·(1−e^{-kt}) (1)

where C(t) is the plasma citrulline concentration at time t (days post-surgery), Min is the minimum citrulline concentration (μmol/L) following resection surgery, Max is the maximum citrulline concentration (μmol/L) following adaptation, and k is the exponential growth rate constant with units of day⁻¹. The decrease from pre-surgery steady-state citrulline levels to the observed minimum was not modeled because sampling within 24 hours of surgery significantly increased animal mortality. However, an initial study of plasma citrulline during this timeframe indicate that plasma citrulline levels following surgery decline in a linear fashion until a minimum is reached at approximately 24 hours (data not shown).

To construct the mixed effects model, interindividual random effects were modeled for all four pharmacodynamic (PD) parameters (Baseline, Min, Max, and k). These effects were modeled using an exponential form described below:
P^{individual}=P^{population}·eη(2)

where P^{individual} is the PD parameter estimate of an individual, P^{population} is the population PD parameter estimate, and η is the interindividual variability estimate for parameter P. Initial model evaluation demonstrated that η estimates for the parameters Min, Max and k were consistent between the resection groups, therefore a single η parameter for each of these 3 PD parameters was estimated. This reduction in the number of parameters in the model improved the model fit and reduced variability in the PD parameter estimates.

The residual variability (ie, random error) was modeled using the heteroscedastic error model described below:
Y=F(1+ε)· (3)

where Y is the observed plasma citrulline concentration, F is the predicted plasma citrulline concentration (equation 1), and ε is the residual variability estimate for the model.

Methods

Male and female Sprague-Dawley rats (71) were assigned to 5 groups (Table I) following a 1-week acclimatization period. Small bowel resection surgery was performed as described below, with the day of surgery considered Day 1. Blood samples for plasma citrulline analysis were obtained 24-48 hours prior to surgery, on Days 2 through 29 (2 samples per calendar week), and just prior to euthanasia on Day 30. All blood samples were obtained from conscious animals by jugular venipuncture into EDTA collection tubes. Plasma was isolated by centrifugation and samples were stored at −70° C. until analysis. Samples were analyzed for citrulline levels with a validated LC-MS/MS method with a lower limit of quantification of 0.86 μmol/L.

Small Bowel Resection Surgery

Prior to surgery, each rat was fasted overnight. The surgical site was prepared and the animals were maintained on isoflurane (1.5 %) anesthesia and on a temperature-controlled heating pad throughout the surgery. A midline abdominal incision was made and the small intestine was exposed. The proximal transection was made distal to the Ligament of Treitz, while the distal transection was made proximal to the ileocecal junction (Table II). At least 10 cm of ileum was retained to ensure survival without supplemental intravenous nutrition. The intestine measurements were made by placing a pre-measured piece of sterile dental tape along the anti-mesenteric border of the gently stretched small intestine. Following ligation of the mesenteric vessels, the resected tissue was removed. Following resection bowel continuity was restored using an end-to-end jejunoileal anastamosis. Bowel continuity was verified and the abdominal cavity was checked for bleeding. The incision was closed using standard techniques. The sham surgery was performed in identical fashion except only a single partial transection, through approximately one-third of the bowel, was made in ileum. The defect was repaired and the incision was closed. Following surgery, animals received water and food (Purified rodent diet AIC-93G, Dyets, Inc.) ad libitum throughout the remainder of the study. Animals did not receive antibiotic treatment to avoid potentially confounding effects.

Results

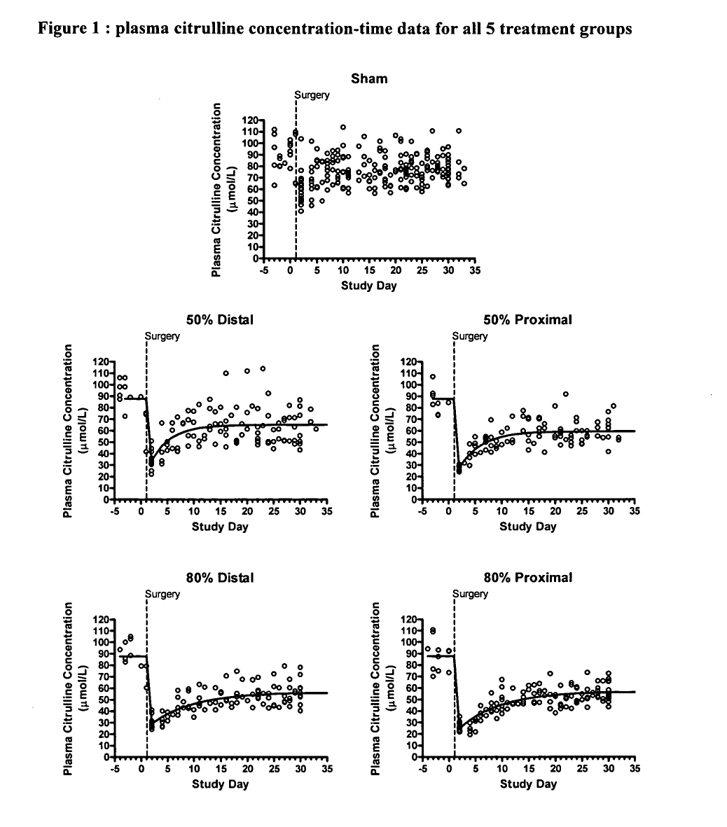

A total of 695 plasma citrulline concentrations samples were collected from 71 rats in this study. Sixty-three (89%) rats survived through Day 30. The individual plasma citrulline concentration data are shown by treatment group in FIG. 1. Observed values shown as open circles. Population predicted mean response from the pharmacodynamic model shown as a solid line. Surgery day (Day 1) indicated with vertical dashed line.

In all groups, there was a decline in plasma citrulline levels followed by an increase to a new steady-state level. The sham group was the only group to return to pre-surgery levels. All 4 small bowel resection groups reached the new steady-state level between approximately 15-25 days following the small bowel resection surgery. The mean plasma citrulline concentrations prior to surgery are presented by treatment group in Table III. No gender differences were observed for any treatment group; therefore data were analyzed by treatment group only.

The sham group demonstrated a slight decrease in plasma citrulline following surgery; however, the decrease was small and recovery to pre-surgery levels occurred within 1 week. Although the plasma citrulline concentration-time data from the sham group could have been fit to the pharmacodynamic model (equation 1), the data was best fit by a line with a slope that was not different from zero (95% CI of the slope is −0.096 to 0.266) suggesting that plasma citrulline was at steady-state in these animals throughout the study. The mean (SD) plasma citrulline concentration for the sham group was 77.41 (14.29) lmol/L, while the range was 41 to 114 μmol/L.

The resection group analysis dataset included 472 plasma citrulline concentration values. The final model included a pre-surgery steady-state parameter (Baseline), 3 PD parameters (Min, Max, k) for each small bowel resection group, 4 interindividual variability parameters (η_{Min}, η_{Max}, ηₖ, η_{Baseline}), and a residual error parameter (η). Final PD parameter estimates and the percent relative standard error (%RSE) of each of those estimates are shown in Table IV. The final variability parameter estimates and %RSE are shown in Table V. The predicted population mean response is shown as a solid line in FIG. 1.

The decrease in plasma citrulline concentrations following small bowel resection surgery is marked for all groups. The rise to a new steady-state level is well characterized by the exponential growth function, and none of the resection groups demonstrate a return to pre-surgery levels. The variability in PD parameter estimates was less than 8% for Baseline, Min and Max and 14-23% for k. Overall, the population PD model accounted for 88% of the variability observed in the plasma citrulline data. Interindividual variability, expressed as percent coefficient of variation (%CV), was low for Baseline (8.8%), Min (12.6%) and Max (15.2%), yet it was large for k (48.3%) suggesting that the growth rate was variable. Even when two interindividual variability parameters were used, one each for the 50% and 80% resection groups, variability estimates were of similar magnitude and the overall model fit was not significantly improved.

Human Study

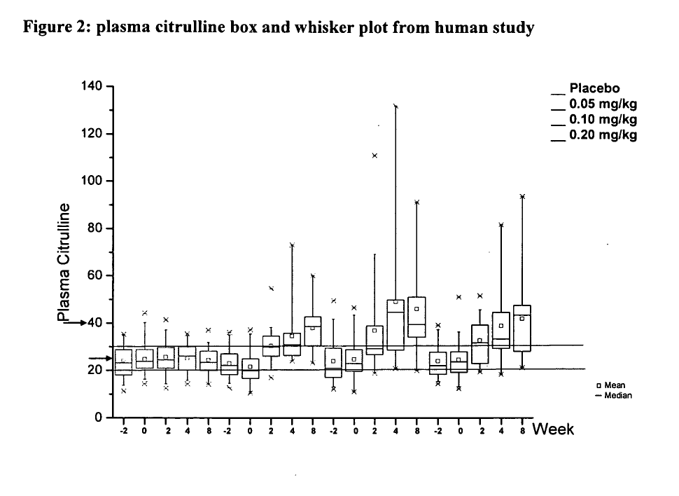

In a further experiment plasma citrulline levels in human patients treated with Teduglutide for the treatment of Crohn's Disease (CD) were determined. Subjects with moderately active CD were treated in a randomized, double-blind, placebo-controlled clinical study to assess the activity of 3 different doses of Teduglutide compared to placebo. The primary objective of the study was to assess the efficacy and the secondary objectives were to assess the safety and tolerability of the different doses of Teduglutide as compared to placebo. Following a screening visit to evaluate whether subjects met the inclusion/exclusion criteria, the study ran over a period of up to 12 weeks, including visits at baseline/Dosing Day 1, Weeks 2, 4, and 8, and one at the end of the follow-up period at Week 12. Teduglutide doses administered by subcutaneous injection were 0.05, 0.10 and 0.20 mg/kg on a daily basis for 8 weeks. The primary efficacy variable used was the percentage of subjects with a response, defined as a 100-point or greater reduction in the subject's CDAI score at dosing Week 8 from their baseline score or a CDAI score less than 150 at Week 8. Plasma citrulline levels are shown in FIG. 2, and tabulated in Tables VI to IX.

Results from Human Study

It can be seen that, from baseline to week 8, the mean citrulline level increases for the teduglutide-treated groups, whilst the placebo group remains the same, indicating that plasma citrulline levels do indeed correlate with response to treatment.

### REFERENCES

Crenn P, Coudray-Lucas C, Cynober L and Messing B (1998) Post-absorptive plasma citrulline concentration: a marker of intestinal failure in humans. Transplant Proc 30:2528.
Crenn P, Coudray-Lucas C, Thuillier F, Cynober L and Messing B (2000) Postabsorptive plasma citrulline concentration is a marker of absorptive enterocyte mass and intestinal failure in humans. Gastroenterology 119:1496-1505.
Crenn P, Vahedi K, Lavergne-Slove A, Cynober L, Matuchansky C and Messing B (2003) Plasma citrulline: A marker of enterocyte mass in villous atrophy-associated small bowel disease. Gastroenterology 124:1210-1219.
Dowling R H (1982) Small bowel adaptation and its regulation. Scand J Gastroenterol Suppl 74:53-74.
Dowling R H and Booth C C (1967) Structural and functional changes following small intestinal resection in the rat. Clin Sci 32:139-149.
Drucker D J (2002) Gut adaptation and the glucagon-like peptides. Gut 50:428-435.
Felig P and Wahren J (1971) Amino acid metabolism in exercising man. J Clin Invest 50:2703-2714.
Flint J (1912) The effect of extensive resections of the small intestine. Bulletin of The Johns Hopkins Hospital 23:127-144.
Hanson W R, Osborne J W and Sharp J G (1977) Compensation by the residual intestine after intestinal resection in the rat. II. Influence of postoperative time interval. Gastroenterology 72:701-705.
Hartmann B, Thulesen J, Hare K J, Kissow H, Orskov C, Poulsen S S and Holst J J (2002) Immunoneutralization of endogenous glucagon-like peptide-2 reduces adaptive intestinal growth in diabetic rats. Regul Pept 105:173-179.
Jianfeng G, Weiming Z, Ning L, Fangnan L, Li T, Nan L and Jieshou L (2005) Serum Citrulline Is A Simple Quantitative Marker for Small Intestinal Enterocytes Mass and Absorption Function in Short Bowel Patients. J Surg Res.
Lardy H, Mouille B, Thomas M, Darcy-Vrillon B, Vaugelade P, Blachier F, Bernard F, Cherbuy C, Robert V, Corriol O, Ricour C, Goulet O, Duee P H and Colomb V (2004) Enterocyte metabolism during early adaptation after extensive intestinal resection in a rat model. Surgery 135:649-656.
Ljungmann K, Hartmann B, Kissmeyer-Nielsen P, Flyvbjerg A, Holst J J and Laurberg S (2001) Time-dependent intestinal adaptation and GLP-2 alterations after small bowel resection in rats. Am J Physiol Gastrointest Liver Physiol 281:G779-785.
Lutgens L C, Blijlevens N M, Deutz N E, Donnelly J P, Lambin P and de Pauw B E (2005) Monitoring myeloablative therapy-induced small bowel toxicity by serum citrulline concentration: a comparison with sugar permeability tests. Cancer 103:191-199.
Lutgens L C, Deutz N, Granzier-Peeters M, Beets-Tan R, De Ruysscher D, Gueulette J, Cleutjens J, Berger M, Wouters B, von Meyenfeldt M and Lambin P (2004) Plasma citrulline concentration: a surrogate end point for radiation-induced mucosal atrophy of the small bowel. A feasibility study in 23 patients. Int J Radiat Oncol Biol Phys 60:275-285.
Lutgens L C, Deutz N E, Gueulette J, Cleutjens J P, Berger M P, Wouters B G, von Meyenfeldt M F and Lambin P (2003) Citrulline: a physiologic marker enabling quantitation and monitoring of epithelial radiation-induced small bowel damage. Int J Radiat Oncol Biol Phys 57:1067-1074.
Perez A, Duxbury M, Rocha F G, Ramsanahie A P, Farivar R S, Varnholt H, Ito H, Wong H, Rounds J, Zinner M J, Whang E E and Ashley S W (2005) Glucagon-like peptide 2 is an endogenous mediator of postresection intestinal adaptation. JPEN J Parenter Enteral Nutr 29:97-101.
Rhoads J M, Plunkett E, Galanko J, Lichtman S, Taylor L, Maynor A, Weiner T, Freeman K, Guarisco J L and Wu GY (2005) Serum citrulline levels correlate with enteral tolerance and bowel length in infants with short bowel syndrome. J Pediatr 146:542-547.
Scott R B, Kirk D, MacNaughton W K and Meddings J B (1998) GLP-2 augments the adaptive response to massive intestinal resection in rat. Am J Physiol 275:G911-921.
Senn N (1888) An experimental contribution to intestinal surgery with special refernce to the treatment of intestinal obstruction, II: enterectomy. Ann. Surg. 7:99-115.
Wasa M, Takagi Y, Sando K, Harada T and Okada A (1999a) Intestinal adaptation in pediatric patients with short-bowel syndrome. Eur J Pediatr Surg 9:207-209.
Wasa M, Takagi Y, Sando K, Harada T and Okada A (1999b) Long-term outcome of short bowel syndrome in adult and pediatric patients. JPEN J Parenter Enteral Nutr 23:S110-112.
Windmueller H G and Spaeth A E (1974) Uptake and metabolism of plasma glutamine by the small intestine. JBiol Chem 249:5070-5079.
Windmueller H G and Spaeth A E (1980) Respiratory fuels and nitrogen metabolism in vivo in small intestine of fed rats. Quantitative importance of glutamine, glutamate, and aspartate. JBiol Chem 255:107-112.

Windmueller H G and Spaeth A E (1981) Source and fate of circulating citrulline. Am J Physiol 241:E473-480.

TABLE I
Small bowel resection groups and number of animals in each group.

| | # of animals |
|---|---|
| Type of small bowel resection | (M/F) |
| Sham | 10/11 |
| 50% Distal | 5/9 |
| 50% Proximal | 5/6 |
| 80% Distal | 5/7 |
| 80% Proximal | 7/6 |
| Total | 32/39 |

TABLE II
Location of transections for small bowel resection groups.

| | Location of | Location of |
|---|---|---|
| | Proximal | Distal |
| Resection Group | Transection^{a} | Transection^{b} |
| 50% Distal | 40 cm | 10 cm |
| 50% Proximal | 0 cm^{c} | 50 cm |
| 80% Distal | 10 cm | 10 cm |
| 80% Proximal | 0 cm^{c} | 20 cm |

^{a}Distal distance from the Ligament of Trietz
^{b}Proximal distance from the ileocecal junction
^{c}Transection performed immediately distal to the Ligament of Trietz

TABLE III
Mean (SD) plasma citrulline concentrations prior to surgery.

| | Mean (SD) | |
|---|---|---|
| Group | (μmol/L) | % CV |
| Sham | 90.6 (15.48) | 17 |
| 50% Distal | 91.2 (11.54) | 13 |
| 50% Proximal | 88.9 (16.62) | 19 |
| 80% Distal | 90.8 (10.02) | 11 |
| 80% Proximal | 88.5 (13.77) | 16 |

TABLE IV
Plasma citrulline PD parameter estimates (% RSE) from mixed effects
model.

| Resection | k | Min | Max | Baseline |
|---|---|---|---|---|
| Group | (day⁻¹) | (μmol/L) | (μmol/L) | (μmol/L) |
| 50% Distal | 0.258 (23.4) | 34.2 (7.11) | 65.3 (6.91) | 87.7 (2.35) |
| 50% Proximal | 0.237 (16.8) | 27.6 (2.59) | 59.5 (4.82) | |
| 80% Distal | 0.123 (14.2) | 29.2 (5.00) | 56.8 (5.26) | |
| 80% Proximal | 0.129 (23.4) | 24.9 (4.42) | 57.1 (4.13) | |

TABLE V
Interindividual variability and percent coefficient of variation (% CV)
from mixed effects model.

| Parameter Name | Estimate | % CV |
|---|---|---|
| η_{Baseline} | 0.008 | 8.8 |
| η_{Max} | 0.023 | 15.2 |
| η_{Min} | 0.016 | 12.6 |
| ηₖ | 0.233 | 48.3 |
| ε | 0.015 | 12.2 |

TABLE VI
Baseline plasma citrulline levels (umol/L) from human study

| Treatment | | | | |
|---|---|---|---|---|
| group | Placebo | 0.05 mg | 0.10 mg | 0.20 mg |
| N | 25 | 22 | 25 | 24 |
| Mean | 24.7 | 21.6 | 24.7 | 24.5 |
| SD | 7.35 | 6.97 | 9.72 | 8.51 |

TABLE VII
2-week plasma citrulline levels (umol/L) from human study

| Treatment | | | | |
|---|---|---|---|---|
| group | Placebo | 0.05 mg | 0.10 mg | 0.20 mg |
| N | 24 | 21 | 20 | 20 |
| Mean | 25.6 | 30.3 | 36.9 | 32.7 |
| SD | 7.18 | 8.17 | 21.04 | 9.61 |

TABLE VIII
4-week plasma citrulline levels (umol/L) from human study

| Treatment | | | | |
|---|---|---|---|---|
| group | Placebo | 0.05 mg | 0.10 mg | 0.20 mg |
| N | 21 | 18 | 16 | 17 |
| Mean | 25.1 | 34.5 | 49.1 | 38.8 |
| SD | 6.28 | 11.67 | 28.54 | 17.93 |

TABLE IX
8-week plasma citrulline levels (umol/L) from human study

| Treatment | | | | |
|---|---|---|---|---|
| group | Placebo | 0.05 mg | 0.10 mg | 0.20 mg |
| N | 20 | 16 | 14 | 18 |
| Mean | 24.3 | 37.9 | 46.0 | 41.8 |
| SD | 6.05 | 9.68 | 21.19 | 16.34 |

1. A method for monitoring improvements in intestinal function in an individual comprising the step of measuring levels of plasma citrulline in the individual over a period of time, wherein an increase in plasma citrulline is indicative of improvement in intestinal function.

2. A method for monitoring the adaptive process of the intestine of an individual comprising the step of monitoring levels of plasma citrulline in the individual over a period of time, wherein an increase in plasma citrulline is indicative of improvement in intestinal function.

3. A method according to claim 1 wherein the individual is undergoing treatment with an analogue of GLP-2.

4. A kit for monitoring the adaptive process of the intestine of an individual by monitoring levels of plasma citrulline in the individual over a period of time comprising reagents and consumables together with instructions for their use.

5. A method according to claim 3 comprising the steps:
a. determining the plasma citrulline level of the subject at a first time point;
b. administering the GLP-2 analogue to the subject; and
c. determining the plasma citrulline level at a second time point;
wherein an increased level of plasma citrulline at the second time point relative to the first time point is indicative of the efficacy of the treatment.

6. A method for determining the efficacy of a test compound administered to a subject for the treatment of intestinal damage, comprising the steps:
a. determining the plasma citrulline level of the subject at a first time point;
b. administering the test compound to the subject; and
c. determining the plasma citrulline level at a second time point
wherein an increased level of plasma citrulline at the second time point relative to the first time point is indicative of the efficacy of the test compound.

7. A method for monitoring the progress of a therapeutic regime designed to treat intestinal damage, comprising:
a. determining the plasma citrulline level of the subject at a first time point;
b. determining the plasma citrulline level at a second time point, wherein the therapeutic regime is followed by the subject between the first and second time points;
wherein an increased level of plasma citrulline at the second time point relative to the first time point is indicative of the efficacy of the treatment regime.
